(19) United States
(12) Patent Application Publication
(10) Pub. No.: US 2014/0058156 A1
(43) Pub. Date: Feb. 27, 2014

(54) PROCESS OF ENERGY MANAGEMENT FROM A METHANE CONVERSION PROCESS

(71) Applicant: UOP LLC, Des Plaines, IL (US)

(72) Inventors: Jeffery C. Bricker, Buffalo Grove, IL (US); John Q. Chen, Glenview, IL (US); Peter K. Coughlin, Mundelein, IL (US)

(21) Appl. No.: 13/966,544

(22) Filed: Aug. 14, 2013

Related U.S. Application Data
(60) Provisional application No. 61/691,377, filed on Aug. 21, 2012.

(51) Int. Cl. C07C 2/76; C01C 1/04

(52) U.S. Cl. CPC C07C 2/76; C01C 1/0488
USPC 585/325; 423/359; 423/362; 423/363

(57) ABSTRACT

Methods and systems are provided for converting methane in a feed stream to acetylene. The method includes heat management in the process for further converting the acetylene stream to form a subsequent hydrocarbon stream. The hydrocarbon stream is introduced into a supersonic reactor and pyrolyzed to convert at least a portion of the methane to acetylene. The reactor effluent stream can be used to transfer heat to process streams used in downstream process units, and in particular streams that are fed to endothermic reactors.

### CROSS REFERENCE TO RELATED APPLICATION

This application claims the benefit of U.S. Provisional Application No. 61/691,377, filed on Aug. 21, 2012.

### FIELD OF THE INVENTION

A process is disclosed for recovering heat during the production of chemicals useful for the production of polymers from the conversion of methane to acetylene using a supersonic flow reactor. More particularly, the process is for the recovery of heat generated during the pyrolysis of methane to acetylene.

### BACKGROUND OF THE INVENTION

The use of plastics and rubbers are widespread in today's world. The production of these plastics and rubbers are from the polymerization of monomers which are generally produced from petroleum. The monomers are generated by the breakdown of larger molecules to smaller molecules which can be modified. The monomers are then reacted to generate larger molecules comprising chains of the monomers. An important example of these monomers are light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries. The main source for these materials in present day refining is the steam cracking of petroleum feeds.

A principal means of production is the cracking of hydrocarbons brought about by heating a feedstock material in a furnace has long been used to produce useful products, including for example, olefin products. For example, ethylene, which is among the more important products in the chemical industry, can be produced by the pyrolysis of feedstocks ranging from light paraffins, such as ethane and propane, to heavier fractions such as naphtha. Typically, the lighter feedstocks produce higher ethylene yields (50-55% for ethane compared to 25-30% for naphtha); however, the cost of the feedstock is more likely to determine which is used. Historically, naphtha cracking has provided the largest source of ethylene, followed by ethane and propane pyrolysis, cracking, or dehydrogenation. Due to the large demand for ethylene and other light olefinic materials, however, the cost of these traditional feeds has steadily increased.

Energy consumption is another cost factor impacting the pyrolytic production of chemical products from various feedstocks. Over the past several decades, there have been significant improvements in the efficiency of the pyrolysis process that have reduced the costs of production. In a typical or conventional pyrolysis plant, a feedstock passes through a plurality of heat exchanger tubes where it is heated externally to a pyrolysis temperature by the combustion products of fuel oil or natural gas and air. One of the more important steps taken to minimize production costs has been the reduction of the residence time for a feedstock in the heat exchanger tubes of a pyrolysis furnace. Reduction of the residence time increases the yield of the desired product while reducing the production of heavier by-products that tend to foul the pyrolysis tube walls. However, there is little room left to improve the residence times or overall energy consumption in tradition pyrolysis processes.

More recent attempts to decrease light olefin production costs include utilizing alternative processes and/or feedstreams. In one approach, hydrocarbon oxygenates and more specifically methanol or dimethylether (DME) are used as an alternative feedstock for producing light olefin products. Oxygenates can be produced from available materials such as coal, natural gas, recycled plastics, various carbon waste streams from industry and various products and by-products from the agricultural industry. Making methanol and other oxygenates from these types of raw materials is well established and typically includes one or more generally known processes such as the manufacture of synthesis gas using a nickel or cobalt catalyst in a steam reforming step followed by a methanol synthesis step at relatively high pressure using a copper-based catalyst.

Once the oxygenates are formed, the process includes catalytically converting the oxygenates, such as methanol, into the desired light olefin products in an oxygenate to olefin (OTO) process. Techniques for converting oxygenates, such as methanol to light olefins (MTO), are described in U.S. Pat. No. 4,387,263, which discloses a process that utilizes a catalytic conversion zone containing a zeolitic type catalyst. U.S. Pat. No. 4,587,373 discloses using a zeolitic catalyst like ZSM-5 for purposes of making light olefins. U.S. Pat. Nos. 5,095,163; 5,126,308 and 5,191,141 on the other hand, disclose an MTO conversion technology utilizing a non-zeolitic molecular sieve catalytic material, such as a metal aluminophosphate (ELAPO) molecular sieve. OTO and MTO processes, while useful, utilize an indirect process for forming a desired hydrocarbon product by first converting a feed to an oxygenate and subsequently converting the oxygenate to the hydrocarbon product. This indirect route of production is often associated with energy and cost penalties, often reducing the advantage gained by using a less expensive feed material. In addition, some oxygenates, such as vinyl acetate or acrylic acid, are also useful chemicals and can be used as polymer building blocks.

Recently, attempts have been made to use pyrolysis to convert natural gas to ethylene. U.S. Pat. No. 7,183,451 discloses heating natural gas to a temperature at which a fraction is converted to hydrogen and a hydrocarbon product such as acetylene or ethylene. The product stream is then quenched to stop further reaction and subsequently reacted in the presence of a catalyst to form liquids to be transported. The liquids ultimately produced include naphtha, gasoline, or diesel. While this method may be effective for converting a portion of natural gas to acetylene or ethylene, it is estimated that this approach will provide only about a 40% yield of acetylene from a methane feed stream. While it has been identified that higher temperatures in conjunction with short residence times can increase the yield, technical limitations prevent further improvement to this process in this regard.

While the foregoing traditional pyrolysis systems provide solutions for converting ethane and propane into other useful hydrocarbon products, they have proven either ineffective or uneconomical for converting methane into these other products, such as, for example ethylene. While MTO technology is promising, these processes can be expensive due to the indirect approach of forming the desired product. Due to continued increases in the price of feeds for traditional processes, such as ethane and naphtha, and the abundant supply and corresponding low cost of natural gas and other methane sources available, for example the more recent accessibility of shale gas, it is desirable to provide commercially feasible and cost effective ways to use methane as a feed for producing ethylene and other useful hydrocarbons.

### SUMMARY OF THE INVENTION

A method for producing acetylene according to one aspect is provided. The method generally includes introducing a feed stream portion of a hydrocarbon stream including methane into a supersonic reactor. The method also includes pyrolyzing the methane in the supersonic reactor to form a reactor effluent stream portion of the hydrocarbon stream including acetylene. The method further includes treating at least a portion of the hydrocarbon stream in a process for producing higher value products.

According to another aspect, a method for controlling a contaminant level in a hydrocarbon stream in the production of acetylene from a methane feed stream is provided. The method includes introducing a feed stream portion of a hydrocarbon stream including methane into a supersonic reactor. The method also includes pyrolyzing the methane in the supersonic reactor to form a reactor effluent stream portion of the hydrocarbon stream including acetylene. The method further includes maintaining the concentration of carbon monoxide in at least a portion of the process stream to below about 100 wt-ppm.

In one embodiment of this invention, the process includes heat integration with other processing units. The invention includes a reaction chamber having a leading section and a trailing section, with the pyrolysis reaction occurring in the leading section to generate a reaction effluent stream. The reaction effluent stream flows to the trailing section where heat from the effluent stream is transferred to a cooling medium. The cooling medium is passed through a heat exchanger disposed within the trailing section, or in cooling tubes that encircle the trailing section of the reaction chamber. The cooling medium is heated and used to add heat to reactors having endothermic processes. The cooling medium can also include feedstreams that are to be preheated.

In one embodiment, the cooling medium is water that is heated to generate steam. The steam can be used to heat reactors, or other process units, or can be used to generate power through a steam turbine. The generation of high temperature steam can also be passed to a high temperature electrolysis unit to generate a hydrogen stream and an oxygen stream.

Other objects, advantages and applications of the present invention will become apparent to those skilled in the art from the following detailed description and drawings.

### BRIEF DESCRIPTION OF THE DRAWINGS

FIG. 1 is a side cross-sectional view of a supersonic reactor in accordance with various embodiments described herein; and

FIG. 2 is a schematic view of a system for converting methane into acetylene and other hydrocarbon products in accordance with various embodiments described herein; and

FIG. 3 is one aspect of utilizing the heat recovery for the production of ammonia.

### DETAILED DESCRIPTION OF THE INVENTION

One proposed alternative to the previous methods of producing hydrocarbon products that has not gained much commercial traction includes passing a hydrocarbon feedstock into a supersonic reactor and accelerating it to supersonic speed to provide kinetic energy that can be transformed into heat to enable an endothermic pyrolysis reaction to occur. Variations of this process are set out in U.S. Pat. Nos. 4,136,015 and 4,724,272, and Russian Patent No. SU 392723A. These processes include combusting a feedstock or carrier fluid in an oxygen-rich environment to increase the temperature of the feed and accelerate the feed to supersonic speeds. A shock wave is created within the reactor to initiate pyrolysis or cracking of the feed. In particular, the hydrocarbon feed to the reactor comprises a methane feed. The methane feed is reacted to generate an intermediate process stream which is then further processed to generate a hydrocarbon product stream. A particular aspect of interest is the energy management of hydrocarbon processes from the formation of higher hydrocarbons from methane.

More recently, U.S. Pat. Nos. 5,219,530 and 5,300,216 have suggested a similar process that utilizes a shock wave reactor to provide kinetic energy for initiating pyrolysis of natural gas to produce acetylene. More particularly, this process includes passing steam through a heater section to become superheated and accelerated to a nearly supersonic speed. The heated fluid is conveyed to a nozzle which acts to expand the carrier fluid to a supersonic speed and lower temperature. An ethane feedstock is passed through a compressor and heater and injected by nozzles to mix with the supersonic carrier fluid to turbulently mix together at a Mach 2.8 speed and a temperature of about 427° C. The temperature in the mixing section remains low enough to restrict premature pyrolysis. The shockwave reactor includes a pyrolysis section with a gradually increasing cross-sectional area where a standing shock wave is formed by back pressure in the reactor due to flow restriction at the outlet. The shock wave rapidly decreases the speed of the fluid, correspondingly rapidly increasing the temperature of the mixture by converting the kinetic energy into heat. This immediately initiates pyrolysis of the ethane feedstock to convert it to other products. A quench heat exchanger then receives the pyrolized mixture to quench the pyrolysis reaction.

Methods and systems for converting hydrocarbon components in methane feed streams using a supersonic reactor are generally disclosed. As used herein, the term “methane feed stream” includes any feed stream comprising methane. The methane feed streams provided for processing in the supersonic reactor generally include methane and form at least a portion of a process stream that includes at least one contaminant. The methods and systems presented herein remove or convert the contaminant in the process stream and convert at least a portion of the methane to a desired product hydrocarbon compound to produce a product stream having a reduced contaminant level and a higher concentration of the product hydrocarbon compound relative to the feed stream. By one approach, a hydrocarbon stream portion of the process stream includes the contaminant and methods and systems presented herein remove or convert the contaminant in the hydrocarbon stream.

The term “hydrocarbon stream” as used herein refers to one or more streams that provide at least a portion of the methane feed stream entering the supersonic reactor as described herein or are produced from the supersonic reactor from the methane feed stream, regardless of whether further treatment or processing is conducted on such hydrocarbon stream. The “hydrocarbon stream” may include the methane feed stream, a supersonic reactor effluent stream, a desired product stream exiting a downstream hydrocarbon conversion process or any intermediate or by-product streams formed during the processes described herein. The hydrocarbon stream may be carried via a process stream line 115, which includes lines for carrying each of the portions of the process stream described above. The term “process stream” as used herein includes the “hydrocarbon stream” as described above, as well as it may include a carrier fluid stream, a fuel stream, an oxygen source stream, or any streams used in the systems and the processes described herein. The process stream may be carried via a process stream line 115, which includes lines for carrying each of the portions of the process stream described above.

Prior attempts to convert light paraffin or alkane feed streams, including ethane and propane feed streams, to other hydrocarbons using supersonic flow reactors have shown promise in providing higher yields of desired products from a particular feed stream than other more traditional pyrolysis systems. Specifically, the ability of these types of processes to provide very high reaction temperatures with very short associated residence times offers significant improvement over traditional pyrolysis processes. It has more recently been realized that these processes may also be able to convert methane to acetylene and other useful hydrocarbons, whereas more traditional pyrolysis processes were incapable or inefficient for such conversions.

The majority of previous work with supersonic reactor systems, however, has been theoretical or research based, and thus has not addressed problems associated with practicing the process on a commercial scale. In addition, many of these prior disclosures do not contemplate using supersonic reactors to effectuate pyrolysis of a methane feed stream, and tend to focus primarily on the pyrolysis of ethane and propane. One problem that has recently been identified with adopting the use of a supersonic flow reactor for light alkane pyrolysis, and more specifically the pyrolysis of methane feeds to form acetylene and other useful products therefrom, includes negative effects that particular contaminants in commercial feed streams can create on these processes and/or the products produced therefrom. Previous work has not considered the need for product purity, especially in light of potential downstream processing of the reactor effluent stream. Product purity can include the separation of several products into separate process streams, and can also include treatments for removal of contaminants that can affect a downstream reaction, and downstream equipment.

In accordance with various embodiments disclosed herein, therefore, processes and systems for converting the methane to a product stream are presented. The methane is converted to an intermediate process stream comprising acetylene. The intermediate process stream is converted to a second process stream comprising either a hydrocarbon product, or a second intermediate hydrocarbon compound. The processing of the intermediate acetylene stream can include purification or separation of acetylene from by-products.

The removal of particular contaminants and/or the conversion of contaminants into less deleterious compounds has been identified to improve the overall process for the pyrolysis of light alkane feeds, including methane feeds, to acetylene and other useful products. In some instances, removing these compounds from the hydrocarbon or process stream has been identified to improve the performance and functioning of the supersonic flow reactor and other equipment and processes within the system. Removing these contaminants from hydrocarbon or process streams has also been found to reduce poisoning of downstream catalysts and adsorbents used in the process to convert acetylene produced by the supersonic reactor into other useful hydrocarbons, for example hydrogenation catalysts that may be used to convert acetylene into ethylene. Still further, removing certain contaminants from a hydrocarbon or process stream as set forth herein may facilitate meeting product specifications.

In accordance with one approach, the processes and systems disclosed herein are used to treat a hydrocarbon process stream, to remove a contaminant therefrom and convert at least a portion of methane to acetylene. The hydrocarbon process stream described herein includes the methane feed stream provided to the system, which includes methane and may also include ethane or propane. The methane feed stream may also include combinations of methane, ethane, and propane at various concentrations and may also include other hydrocarbon compounds. In one approach, the hydrocarbon feed stream includes natural gas. The natural gas may be provided from a variety of sources including, but not limited to, gas fields, oil fields, coal fields, fracking of shale fields, biomass, and landfill gas. In another approach, the methane feed stream can include a stream from another portion of a refinery or processing plant. For example, light alkanes, including methane, are often separated during processing of crude oil into various products and a methane feed stream may be provided from one of these sources. These streams may be provided from the same refinery or different refinery or from a refinery off gas. The methane feed stream may include a stream from combinations of different sources as well.

In accordance with the processes and systems described herein, a methane feed stream may be provided from a remote location or at the location or locations of the systems and methods described herein. For example, while the methane feed stream source may be located at the same refinery or processing plant where the processes and systems are carried out, such as from production from another on-site hydrocarbon conversion process or a local natural gas field, the methane feed stream may be provided from a remote source via pipelines or other transportation methods. For example a feed stream may be provided from a remote hydrocarbon processing plant or refinery or a remote natural gas field, and provided as a feed to the systems and processes described herein. Initial processing of a methane stream may occur at the remote source to remove certain contaminants from the methane feed stream. Where such initial processing occurs, it may be considered part of the systems and processes described herein, or it may occur upstream of the systems and processes described herein. Thus, the methane feed stream provided for the systems and processes described herein may have varying levels of contaminants depending on whether initial processing occurs upstream thereof

In one example, the methane feed stream has a methane content ranging from about 65 mol-% to about 100 mol-%. In another example, the concentration of methane in the hydrocarbon feed ranges from about 80 mol-% to about 100 mol-% of the hydrocarbon feed. In yet another example, the concentration of methane ranges from about 90 mol-% to about 100 mol-% of the hydrocarbon feed.

In one example, the concentration of ethane in the methane feed ranges from about 0 mol-% to about 35 mol-% and in another example from about 0 mol-% to about 10 mol-%. In one example, the concentration of propane in the methane feed ranges from about 0 mol-% to about 5 mol-% and in another example from about 0 mol-% to about 1 mol-%.

The methane feed stream may also include heavy hydrocarbons, such as aromatics, paraffinic, olefinic, and naphthenic hydrocarbons. These heavy hydrocarbons if present will likely be present at concentrations of between about 0 mol-% and about 100 mol-%. In another example, they may be present at concentrations of between about 0 mol-% and 10 mol-% and may be present at between about 0 mol-% and 2 mol-%.

In one embodiment, the present invention includes a process for extracting heat from a methane to acetylene conversion process. The methane is reacted in a supersonic flow reactor to generate a first effluent mixture comprising acetylene. The reaction is a pyrolysis reaction that occurs at very high temperatures over a very short time period. The heat generated is then extracted from the effluent stream in the reactor. The process includes extracting heat from a portion of the reaction chamber in the supersonic flow reactor. The reaction chamber includes a leading section and a trailing section, wherein the extraction of heat comprises cooling the first effluent mixture as it is passed to the trailing section of the reaction chamber. The trailing section of the reaction chamber includes a heat exchange unit disposed around the trailing section of the reaction chamber. The leading section of the reaction chamber can be between 10% and 90% of the reaction chamber, and the trailing portion of the reaction chamber can be between 10% and 90% of the reaction chamber. The split of the reaction chamber can be designed to accommodate the reaction to the extent desired with methane consumed, and the amount of heat removed from the trailing portion of the reaction chamber needed.

The trailing portion of the reaction chamber can be encircled with cooling tubes, with the reaction effluent from the leading portion of the reaction chamber contacting the cooling tubes. The tubes can be lined with a high heat transfer material such as copper, to facilitate heat transfer from the reaction effluent to the cooling tubes. A cooling medium is passed through the cooling tubes to transfer the heat to another hydrocarbon processing unit.

In one embodiment, a feedstream to a hydrocarbon processing unit is preheated by passing the feedstream through the cooling tubes. In particular, the feedstream passed through the cooling tubes comprises a feed to an endothermic reactor, such as a hydrogenation unit, or aromatization and cyclization unit.

In one embodiment, the trailing portion of the reaction chamber can include a heat exchanger unit disposed within the trailing portion of the reaction chamber. Heat is transferred from the reaction effluent stream to the heat transfer medium in the heat exchanger to carry the heat to a downstream hydroprocessing unit. One aspect of this embodiment includes the heat transfer to a dehydrogenation reactor to maintain the temperature in the dehydrogenation reactor during the dehydrogenation reaction process. Another aspect of this embodiment includes the heat transfer to an aromatization reactor to maintain the temperature in the aromatization reactor during the aromatization reaction process. Another aspect of this embodiment includes the heat transfer to a vinyl chloride reactor to maintain the temperature in the vinyl chloride reactor during the vinyl chloride reaction process.

In one embodiment, the process includes passing the methane feedstream through the heat exchanger, or cooling tubes, in the trailing portion of the reaction chamber to preheat the methane feedstream to the supersonic reactor. This embodiment can include splitting the methane feedstream and passing a first portion of the methane feedstream to the reaction and preheating a second portion of the feedstream through the heat exchanger in the trailing portion of the reaction chamber.

In one embodiment, the process includes passing water, or low temperature steam, through the heat exchanger, or through the cooling tubes, to generate a high temperature steam. The steam can then be used in downstream processes, or in other processes requiring the addition of heat. In an alternative, the stream can be passed through steam turbines to convert the heat to power.

In one embodiment, the process includes passing water, or low temperature steam, through the heat exchanger, or cooling tubes, to generate a high temperature steam, and particularly over 700° C. The stream can be used in a high temperature electrolysis unit to generate a hydrogen stream and an oxygen stream. The hydrogen can partly be used in hydrogenation reactors or other processing units that consume hydrogen. The hydrogen and oxygen can partly be passed to a combustion unit. This is particularly useful if the supersonic flow reactor is located in a location where there is a low availability of an enriched oxygen source.

In one embodiment of the present invention includes the ability to make ammonia for subsequent processes. The production of ammonia requires high temperatures to obtain satisfactory yields. Ammonia production is important for a wide range of chemicals, and especially fertilizers, which can consume as much as 1 to 2% of world wide fossil fuel energy consumption. The present invention utilizes the large amount of heat at high temperatures generated in the supersonic reactor to produce ammonia for the generation of downstream chemicals where ammonia is a precursor. The process includes recovering hydrogen from reactor effluent stream and passing the hydrogen with a source of nitrogen to an ammonia reactor. The heat for the ammonia reactor can be supplied by the supersonic reactor through known heat transfer means. The heat can also be passed to the reactor through passing the hydrogen and nitrogen feedstreams through heating coils, either in or surrounding the reaction chamber of the supersonic reactor.

The method for ammonia production, and heat recovery includes reacting a methane feed in a supersonic reactor to convert the methane to acetylene in an effluent stream. The effluent stream is passed to a separation unit to generate a first stream comprising acetylene, and a second stream comprising hydrogen. The second stream and a nitrogen stream are passed to an ammonia reactor, where heat is supplied from the supersonic reactor.

The ammonia reactor includes a catalyst, and is operated at a temperature between 300° C. and 550° C. The ammonia reactor conditions include a pressure between 15 and 25 MPa, and the nitrogen source can be air, or a nitrogen enriched source. The catalyst in the ammonia reactor includes a metal or metal oxide on a support. The metal, or metal oxide, can be selected from iron, osmium, or ruthenium, and can also include a mixture of metals. The catalyst can also include a promoter, wherein the promoter is selected from K2O, CaO, SiO2, and Al2O3. The promoter can also be a part of the support, or can be a mixture added to the metal or metal oxide on a support

The process for forming acetylene from the methane feed stream described herein utilizes a supersonic flow reactor for pyrolyzing methane in the feed stream to form acetylene. The supersonic flow reactor may include one or more reactors capable of creating a supersonic flow of a carrier fluid and the methane feed stream and expanding the carrier fluid to initiate the pyrolysis reaction. In one approach, the process may include a supersonic reactor as generally described in U.S. Pat. No. 4,724,272, which is incorporated herein by reference, in their entirety. In another approach, the process and system may include a supersonic reactor such as described as a “shock wave” reactor in U.S. Pat. Nos. 5,219,530 and 5,300,216, which are incorporated herein by reference, in their entirety. In yet another approach, the supersonic reactor described as a “shock wave” reactor may include a reactor such as described in “Supersonic Injection and Mixing in the Shock Wave Reactor” Robert G. Cerff, University of Washington Graduate School, 2010.

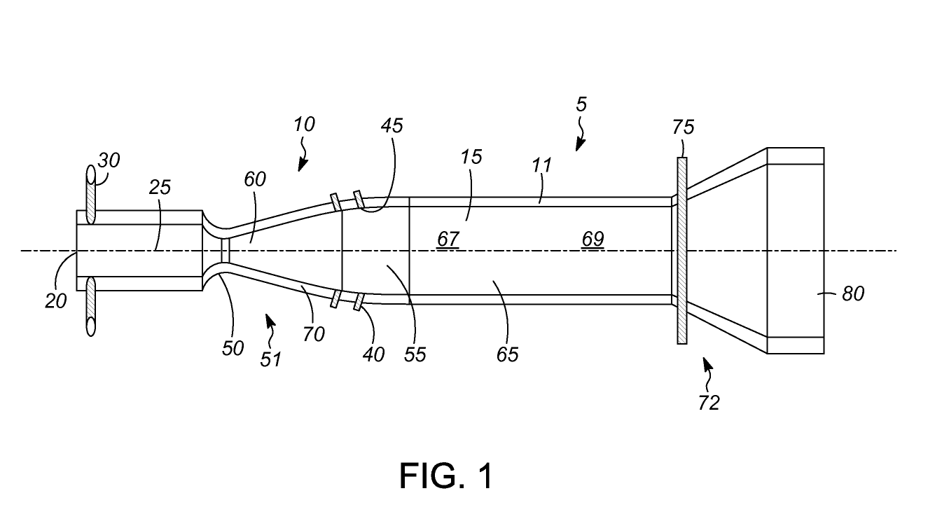

While a variety of supersonic reactors may be used in the present process, an exemplary reactor 5 is illustrated in FIG. 1. Referring to FIG. 1, the supersonic reactor 5 includes a reactor vessel 10 generally defining a reactor chamber 15. While the reactor 5 is illustrated as a single reactor, it should be understood that it may be formed modularly or as separate vessels. A combustion zone or chamber 25 is provided for combusting a fuel to produce a carrier fluid with the desired temperature and flowrate. The reactor 5 may optionally include a carrier fluid inlet 20 for introducing a supplemental carrier fluid into the reactor. One or more fuel injectors 30 are provided for injecting a combustible fuel, for example hydrogen, into the combustion chamber 25. The same or other injectors may be provided for injecting an oxygen source into the combustion chamber 25 to facilitate combustion of the fuel. The fuel and oxygen are combusted to produce a hot carrier fluid stream typically having a temperature of from about 1200° C. to about 3500° C. in one example, between about 2000° C. and about 3500° C. in another example, and between about 2500° C. and 3200° C. in yet another example. According to one example the carrier fluid stream has a pressure of about 100 kPa or higher, greater than about 200 kPa in another example, and greater than about 400 kPa in another example.

The hot carrier fluid stream from the combustion zone 25 is passed through a converging-diverging nozzle 50 to accelerate the flowrate of the carrier fluid to above about mach 1.0 in one example, between about mach 1.0 and mach 4.0 in another example, and between about mach 1.5 and 3.5 in another example. In this regard, the residence time of the fluid in the reactor portion of the supersonic flow reactor is between about 0.5 to 100 ms in one example, about 1 to 50 ms in another example, and about 1.5 to 20 ms in another example.

A feedstock inlet 40 is provided for injecting the methane feed stream into the reactor 5 to mix with the carrier fluid. The feedstock inlet 40 may include one or more injectors 45 for injecting the feedstock into the nozzle 50, a mixing zone 55, an expansion zone 60, or a reaction zone or chamber 65. The injector 45 may include a manifold, including for example a plurality of injection ports. In heat recovery, the reaction chamber 65 can be divided into two zones, a leading zone 67 and a trailing zone 69, wherein the reaction primarily takes place in the leading zone 67 and the temperature is high and a reaction product is generated. As the reaction product moves down the reaction chamber 65 from the leading zone 67 to the trailing zone 69, the reaction product can be cooled. Control parameters and the time allowed for the reaction will determine the relative sizes of the leading zone 67 and the trailing zone 69. The trailing zone 69 can include cooling tubes encircling the trailing zone 69, or other means for transferring heat from the reaction product out of the trailing zone 69 of the reaction chamber 65. Other means can include a heat exchanger with the reaction product flowing through the heat exchanger, a series of high conductivity fins extending into the zone 69 including fins extending off of cooling tubes, or other means that contact the reaction product with the heat transfer mechanism.

In one approach, the reactor 5 may include a mixing zone 55 for mixing of the carrier fluid and the feed stream. In another approach, no mixing zone is provided, and mixing may occur in the nozzle 50, expansion zone 60, or reaction zone 65 of the reactor 5. An expansion zone 60 includes a diverging wall 70 to produce a rapid reduction in the velocity of the gases flowing therethrough, to convert the kinetic energy of the flowing fluid to thermal energy to further heat the stream to cause pyrolysis of the methane in the feed, which may occur in the expansion section 60 and/or a downstream reaction section 65 of the reactor. The fluid is quickly quenched in a quench zone 72 to stop the pyrolysis reaction from further conversion of the desired acetylene product to other compounds. Spray bars 75 may be used to introduce a quenching fluid, for example water or steam into the quench zone 72.

The reactor effluent exits the reactor via outlet 80 and as mentioned above forms a portion of the hydrocarbon stream. The effluent will include a larger concentration of acetylene than the feed stream and a reduced concentration of methane relative to the feed stream. The reactor effluent stream may also be referred to herein as an acetylene stream as it includes an increased concentration of acetylene. The acetylene may be an intermediate stream in a process to form another hydrocarbon product or it may be further processed and captured as an acetylene product stream. In one example, the reactor effluent stream has an acetylene concentration prior to the addition of quenching fluids ranging from about 2 mol-% to about 30 mol-%. In another example, the concentration of acetylene ranges from about 5 mol-% to about 25 mol-% and from about 8 mol-% to about 23 mol-% in another example.

In one example, the reactor effluent stream has a reduced methane content relative to the methane feed stream ranging from about 15 mol-% to about 95 mol-%. In another example, the concentration of methane ranges from about 40 mol-% to about 90 mol-% and from about 45 mol-% to about 85 mol-% in another example.

In one example the yield of acetylene produced from methane in the feed in the supersonic reactor is between about 40 mol-% and about 95 mol-%. In another example, the yield of acetylene produced from methane in the feed stream is between about 50 mol-% and about 90 mol-%. Advantageously, this provides a better yield than the estimated 40% yield achieved from previous, more traditional, pyrolysis approaches.

By one approach, the reactor effluent stream is reacted to form another hydrocarbon compound. In this regard, the reactor effluent portion of the hydrocarbonstream may be passed from the reactor outlet to a downstream hydrocarbon conversion process for further processing of the stream. While it should be understood that the reactor effluent stream may undergo several intermediate process steps, such as, for example, water removal, adsorption, and/or absorption to provide a concentrated acetylene stream, these intermediate steps will not be described in detail herein.

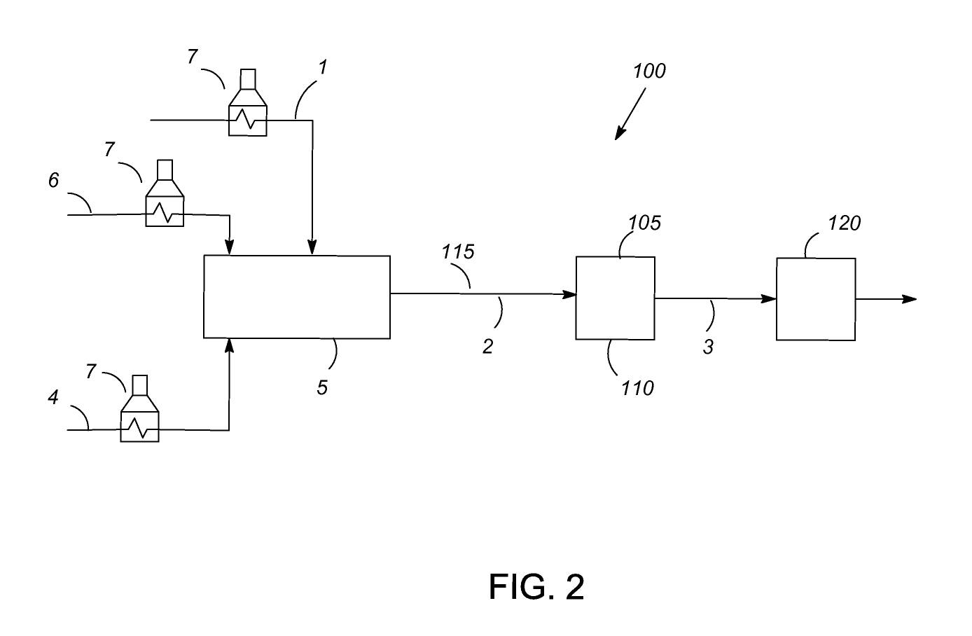

Referring to FIG. 2, the reactor effluent stream having a higher concentration of acetylene may be passed to a downstream hydrocarbon conversion zone 100 where the acetylene may be converted to form another hydrocarbon product. The hydrocarbon conversion zone 100 may include a hydrocarbon conversion reactor 105 for converting the acetylene to another hydrocarbon product. While FIG. 2 illustrates a process flow diagram for converting at least a portion of the acetylene in the effluent stream to ethylene through hydrogenation in hydrogenation reactor 110, it should be understood that the hydrocarbon conversion zone 100 may include a variety of other hydrocarbon conversion processes instead of or in addition to a hydrogenation reactor 110, or a combination of hydrocarbon conversion processes. Similarly, it illustrated in FIG. 2 may be modified or removed and are shown for illustrative purposes and not intended to be limiting of the processes and systems described herein. Specifically, it has been identified that several other hydrocarbon conversion processes, other than those disclosed in previous approaches, may be positioned downstream of the supersonic reactor 5, including processes to convert the acetylene into other hydrocarbons, including, but not limited to: alkenes, alkanes, methane, acrolein, acrylic acid, acrylates, acrylamide, aldehydes, polyacetylides, benzene, toluene, styrene, aniline, cyclohexanone, caprolactam, propylene, butadiene, butyne diol, butandiol, C2-C4 hydrocarbon compounds, ethylene glycol, diesel fuel, diacids, diols, pyrrolidines, and pyrrolidones.

A contaminant removal zone 120 for removing one or more contaminants from the hydrocarbon or process stream may be located at various positions along the hydrocarbon or process stream depending on the impact of the particular contaminant on the product or process and the reason for the contaminants removal, as described further below. For example, particular contaminants have been identified to interfere with the operation of the supersonic flow reactor 5 and/or to foul components in the supersonic flow reactor 5. Thus, according to one approach, a contaminant removal zone is positioned upstream of the supersonic flow reactor in order to remove these contaminants from the methane feed stream prior to introducing the stream into the supersonic reactor. Other contaminants have been identified to interfere with a downstream processing step or hydrocarbon conversion process, in which case the contaminant removal zone may be positioned upstream of the supersonic reactor or between the supersonic reactor and the particular downstream processing step at issue. Still other contaminants have been identified that should be removed to meet particular product specifications. Where it is desired to remove multiple contaminants from the hydrocarbon or process stream, various contaminant removal zones may be positioned at different locations along the hydrocarbon or process stream. In still other approaches, a contaminant removal zone may overlap or be integrated with another process within the system, in which case the contaminant may be removed during another portion of the process, including, but not limited to the supersonic reactor 5 or the downstream hydrocarbon conversion zone 100. This may be accomplished with or without modification to these particular zones, reactors or processes. While the contaminant removal zone 120 illustrated in FIG. 2 is shown positioned downstream of the hydrocarbon conversion reactor 105, it should be understood that the contaminant removal zone 120 in accordance herewith may be positioned upstream of the supersonic flow reactor 5, between the supersonic flow reactor 5 and the hydrocarbon conversion zone 100, or downstream of the hydrocarbon conversion zone 100 as illustrated in FIG. 2 or along other streams within the process stream, such as, for example, a carrier fluid stream, a fuel stream, an oxygen source stream, or any streams used in the systems and the processes described herein.

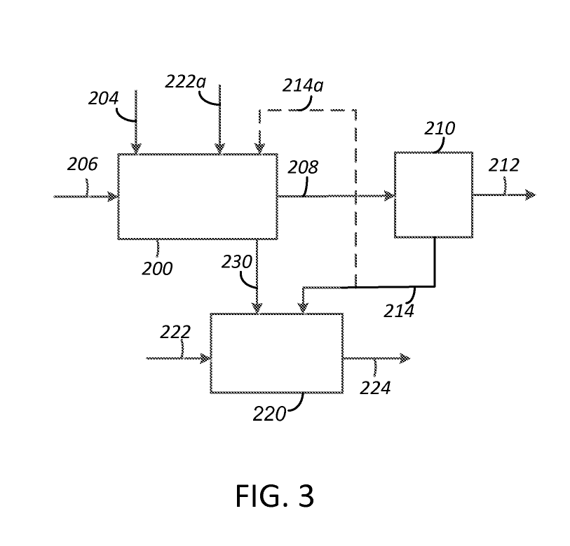

While there are many processes that can utilize the energy, one process where energy is an important concern is the production of ammonia. The present invention in this embodiment is shown in FIG. 3, wherein a methane stream 204 is passed to a supersonic reactor unit 200. The unit 200 includes a feed of fuel 206, usually hydrogen and oxygen, for generating the supersonic flow. The reactor unit 200 pyrolyzes the methane to generate a reactor effluent stream 208 comprising acetylene, CO and H2. The effluent stream 208 is processed in a separation zone 220 to generate an acetylene stream 212 and a hydrogen stream 214. The acetylene stream 214 is passed to a second reactor unit (not shown) for further processing. The hydrogen stream 214 is passed to an ammonia reactor 220, along with a nitrogen stream 222 to generate an ammonia stream 224. Heat is transferred from the reactor unit 200 to the ammonia reactor 220 through a heat transfer means 230. One means of transferring the heat is to pass the hydrogen stream 214 through a line 214a to heat the hydrogen before passing the hydrogen to the ammonia reactor. In a similar manner, nitrogen, or air, can be heated 222a through the reactor unit 200.

While there have been illustrated and described particular embodiments and aspects, it will be appreciated that numerous changes and modifications will occur to those skilled in the art, and it is intended in the appended claims to cover all those changes and modifications which fall within the true spirit and scope of the present disclosure and appended claims.

What is claimed is:

1. A method to recover heat from a supersonic flow reactor, comprising:
reacting methane in a supersonic flow reactor to form a first effluent mixture comprising acetylene, CO and H2, and heat;
passing the first effluent mixture to a second reactor to form a second effluent mixture; and
extracting the heat from the supersonic flow reactor.

2. The method of claim 1 wherein the supersonic flow reactor includes a reaction chamber with a leading section of the chamber and a trailing section of the chamber, and wherein the extraction of heat comprises cooling the first effluent mixture in the trailing section of the reaction chamber, wherein the trailing second of the reaction chamber comprises a heat exchange unit disposed around the trailing section of the reaction chamber.

3. The method of 2 wherein the cooling of the first effluent mixture comprises contacting the first effluent mixture with cooling tubes disposed within the trailing section of the reaction chamber.

4. The method of claim 3 wherein a cooling medium is passed through the cooling tubes.

5. The method of claim 3 wherein a feed to a dehydrogenation reactor is passed through the cooling tubes.

6. The method of claim 3 wherein a feed to a reactor for cyclization and aromatization of a hydrocarbon stream is passed through the cooling tubes.

7. The method of claim 2 wherein the trailing section of the reaction chamber includes a heat exchanger.

8. The method of claim 7 wherein a heat exchange fluid is passed between the heat exchanger in the trailing section of the reaction chamber and passed to a second heat exchanger used to heat downstream reactors.

9. The method of claim 2 further comprising passing water through the heat exchanger of the trailing section of the reaction chamber to generate a steam stream.

10. The method of claim 2 further comprising preheating the methane through a heat exchanger disposed in the trailing section of the reaction chamber.

11. The method of claim 2 further comprising splitting the methane into a first portion, and a second portion, wherein the first portion is fed to the supersonic reactor, and the second portion is passed to a heat exchanger disposed in the trailing section of the reaction chamber.

12. A method of recovering heat from a supersonic flow reactor wherein the reactor comprises a chamber having a leading section and a trailing section, comprising:
passing a methane feedstream through a heat exchange unit disposed around the trailing section of the reaction chamber, to generate a preheated methane stream;
passing the preheated methane stream to the reactor at a methane inlet disposed upstream of the leading section of the reaction chamber;
reacting methane in a supersonic flow reactor to form a first effluent mixture comprising acetylene, CO and H2, and heat; and
passing the first effluent mixture across the heat exchange unit to generate a cooled first effluent mixture.

13. The method of claim 12 further comprising passing the cooled effluent mixture to a second reaction unit comprising a hydrocarbon conversion process for converting acetylene to a second process stream.

14. The method of claim 13 wherein the second reaction unit is a hydrogenation unit to generate an olefin stream comprising ethylene.

15. The method of claim 14 further comprising passing the olefin stream to an oligomerization unit to generate an olefin stream comprising C4+ olefins.

16. A method to recover heat for the production of ammonia from a supersonic flow reactor, comprising:
reacting methane in a supersonic flow reactor to form a first effluent mixture comprising acetylene, CO and H2, and heat;
passing the first effluent mixture to a separation unit to form a first process stream comprising acetylene and a second process stream comprising hydrogen;
extracting the heat from the supersonic flow reactor and passing the heat to an ammonia reactor; and
passing the second process stream and a nitrogen process stream to the ammonia reactor to generate an ammonia effluent stream.

17. The method of claim 16 wherein the ammonia reactor is heated to a temperature between 300° C. and 550° C. and is operated at a pressure between 15 and 25 MPa.

18. The method of claim 16 wherein the ammonia reactor includes a catalyst comprising a metal or a metal oxide, where the metal is selected from the group consisting of iron, osmium, ruthenium, and mixtures thereof

19. The method of claim 18 wherein the catalyst includes a promoter selected from the group consisting of K2O, CaO, SiO2, Al2O3, and mixtures thereof.

20. The method of claim 19 wherein the catalyst includes a support.
